(19) United States
(12) Patent Application Publication
(10) Pub. No.: US 2018/0000379 A1
(43) Pub. Date: Jan. 4, 2018

(54) Patient Reminder System and Method for Incentive Spirometer Utilization

(71) Applicant: BROWN UNIVERSITY, PROVIDENCE, RI (US)

(72) Inventors: ADAM ELTORAI, PROVIDENCE, RI (US); Jeffrey KARG, Bolton, MA (US); Hernando Cenon VIDAL, JR., Englewood Cliffs, NJ (US); Peter MCHUGH, Boston, MA (US)

(21) Appl. No.: 15/593,893

(22) Filed: May 12, 2017

Related U.S. Application Data
(60) Provisional application No. 62/335,100, filed on May 12, 2016.

(51) Int. Cl. A61B 5/091; A61B 5/00

(52) U.S. Cl. CPC A61B 5/091; A61B 5/7475; A61B 5/486; A61B 5/72; A61M 2230/40

(57) ABSTRACT

A system and method for configuring and/or utilizing an incentive spirometer to interact with a user during a series of sessions, the incentive spirometer having a piston that is movable within a chamber by user inhalation. A sensor is mounted to detect movement of the piston, a perceptible prompt to a user is generated to encourage use of the incentive spirometer at preselected intervals, and the perceptible prompt is deactivated only after the sensor detects that the piston has at least reached a preselected threshold.

### CROSS-REFERENCE TO RELATED APPLICATION

This application claims priority to U.S. Provisional Application No. 62/335,100 filed on 12 May 2016. The entire contents of the above-mentioned application is incorporated herein by reference.

### FIELD OF THE INVENTION

This invention relates to respiratory therapy devices and more particularly to devices and methods encouraging patients to properly utilize incentive spirometers.

### BACKGROUND OF THE INVENTION

A commonly recommended technique for post-operative care utilizes incentive spirometers to exercise the lungs to enhance respiration capabilities of patients. As noted by many including Weinstein et al. in U.S. Pat. No. 6,238,353, it is often difficult to monitor actual usage of an incentive spirometer by a patient. Patient forgetfulness is a major concern. Poor incentive spirometer compliance can lead to various clinical problems such as atelectasis, pneumonia or other costly postoperative pulmonary complications in patients.

It is therefore desirable to encourage actual and effective use of incentive spirometers without interfering with the accuracy of the manner in which the device is utilized.

### SUMMARY OF THE INVENTION

An object of the present invention is to remind a patient to engage in successive sessions utilizing a device such as an incentive spirometer.

Another object of the present invention is to ensure that a patient achieves sufficient inspiratory target volumes during each session.

This invention features a method for configuring and/or utilizing an incentive spirometer to interact with a user during a series of sessions, the incentive spirometer having a piston that is movable within a chamber by user inhalation. The method includes mounting a sensor to detect movement of the piston, generating a perceptible prompt to a user to encourage use of the incentive spirometer at preselected intervals relative to the series of sessions, and deactivating the perceptible prompt only after the sensor detects that the piston has at least reached a preselected threshold.

In one embodiment, the perceptible prompt includes an audible sound. In another embodiment, the perceptible prompt includes a visual signal, such as a blinking light. In some embodiments, the perceptible prompt includes a tactile signal, such as a vibratory sensation. In certain embodiments, the perceptible prompt include a combination signals. In another embodiment, generating the perceptible prompt includes activating a prompt on a remote device (such as a mobile device capable of being carried by the user and/or a clinician or other health care professional), and/or reading data output and/or recording data output. In a number of embodiments, the method further includes storing detected piston movement in a storage medium as retrievable data. In some embodiments, the sensor is removably mounted to the incentive spirometer such as being integrated into a removable clamp. In certain embodiments, the perceptible prompt is deactivated only after the user breathes to sufficient inspiratory target volume into the incentive spirometer. In certain embodiments, the system further includes an opaque housing that covers at least a portion of the incentive spirometer housing. In some embodiments, the sensor is capable of detecting optical radiation transmitted through the housing of the incentive spirometer, and the opaque housing is secured to a support base and is positioned to shield at least a portion of environmental optical radiation surrounding the incentive spirometer housing.

This invention also features a system to enhance compliance for a user of an incentive spirometer having a piston that is movable within a chamber by user inhalation. The system includes a sensor mountable on the incentive spirometer to detect movement of the piston, and an alarm device for generating a perceptible prompt to the user to encourage use of the incentive spirometer. The system further includes a processor capable of periodically activating the alarm device and then deactivating the alarm device only after the sensor detects that the piston has at least reached a preselected threshold.

### BRIEF DESCRIPTION OF THE DRAWINGS

In what follows, preferred embodiments of the invention are explained in more detail with reference to the drawings, in which:

FIG. 1 is a schematic diagram showing a system according to the present invention sensing movement of a piston on an incentive spirometer and wirelessly communicating with a laptop;

FIG. 2 is a schematic diagram of a sensor array with multiple detectors utilized according to the present invention to detect piston movement;

FIG. 3 is a schematic diagram of another sensor utilized according to the present invention to detect vertical distance of the piston from the sensor and volume of displaced air;

FIG. 4 is a schematic diagram of an alternative circuit design;

FIG. 5 is a schematic top view of one system according to the present invention integrated into a clamp that is removably placed on an incentive spirometer;

FIG. 6 is an operational flow chart for one system according to the present invention;

FIG. 7 is a schematic rendering of oscilloscope readings as a piston passes up (lower arrows) and down (upper arrows);

FIG. 8A is a representative flow chart of system operation, and FIGS. 8B and 8C are representative examples of alternative state control diagrams of a host laptop and embedded computer, respectively;

FIG. 9 is a representative example of listing of command lines for firmware in one system according to the present invention;

FIGS. 10A and 10D are schematic perspective views of an apparatus with an opaque housing for one system according to the present invention;

FIG. 10B is a schematic perspective view of the support base of FIGS. 10A and 10D;

FIG. 10C is a schematic view within the sensor positioner illustrated in FIGS. 10A and 10D;

FIG. 10E is a schematic perspective view of certain electronics for the apparatus of FIGS. 10A and 10D; and

FIG. 11 is a schematic diagram of a hardware system configuration for the system of FIGS. 10A-10E.

### DETAILED DESCRIPTION OF THE PRESENTLY PREFERRED EMBODIMENTS

This invention may be accomplished by a system and method for configuring and/or utilizing an incentive spirometer to interact with a user during a series of sessions, the incentive spirometer having a piston that is movable within a chamber by user inhalation. The method includes mounting a sensor to detect movement of the piston, generating a perceptible prompt to a user to encourage use of the incentive spirometer at preselected intervals relative to the series of sessions, and deactivating the perceptible prompt only after the sensor detects that the piston has at least reached a preselected threshold.

In one construction, a system according to the present invention includes a sensor mountable on the incentive spirometer to detect movement of the piston, and an alarm device for generating a perceptible prompt, such as an audible sound, to the user to remind and encourage use of the incentive spirometer. The system further includes a processor capable of periodically activating the alarm device, such as when a session should be commenced, and then deactivating the alarm device only after the sensor detects that the piston has at least reached a preselected threshold inspiratory volume, such as after the user inhales with sufficient inspiratory target volume from the device during that session, and successfully inhales one or more times to a predetermined volume.

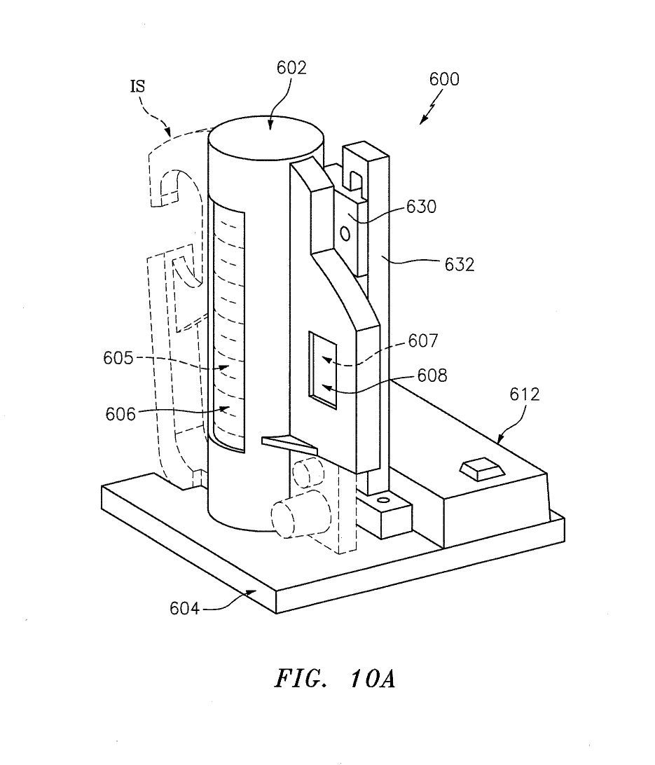

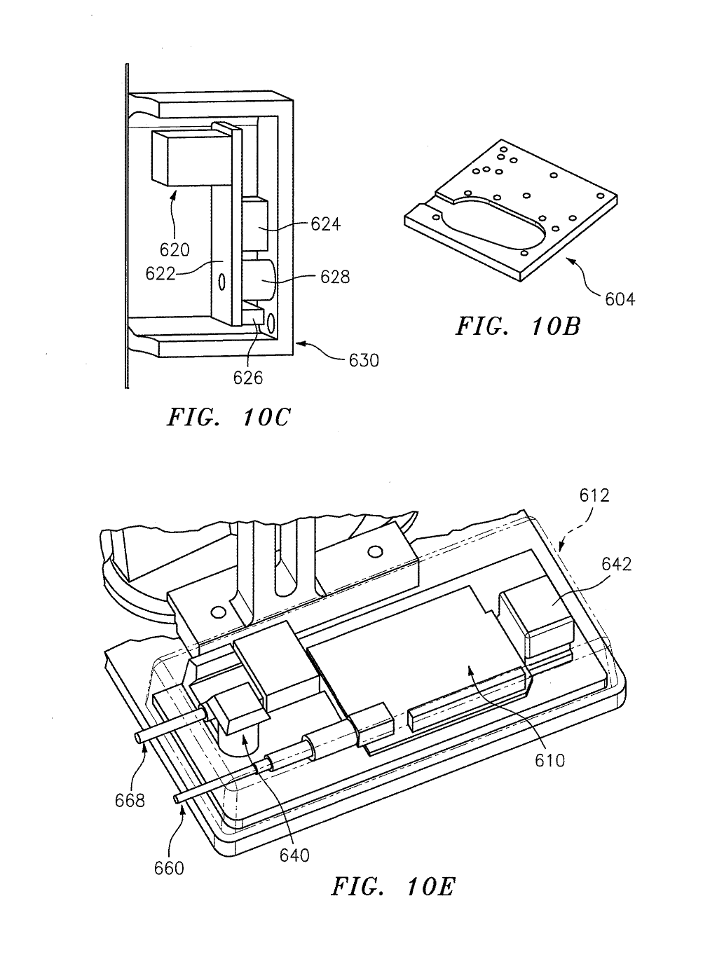

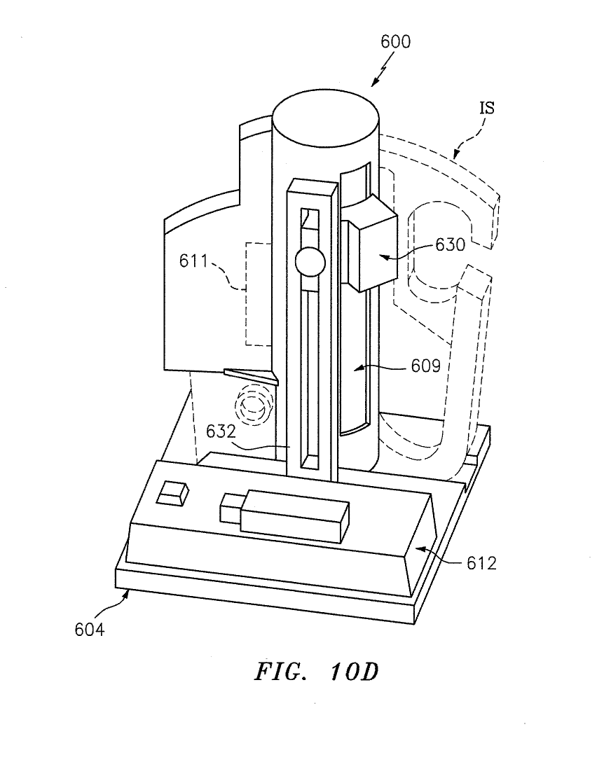

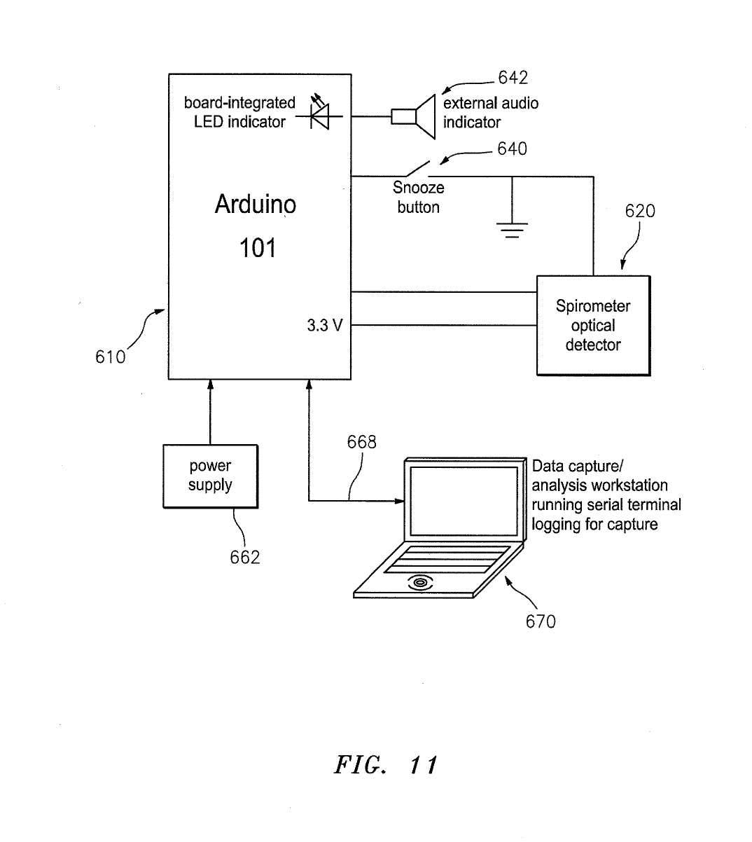

In preferred constructions, the patient reminder alarm only turns off when the patient utilizes the incentive spirometer as prescribed by a healthcare professional. In certain constructions, the system includes an opaque housing that covers at least a portion of the incentive spirometer housing, as discussed in more detail below in relation to FIG. 10A onward.

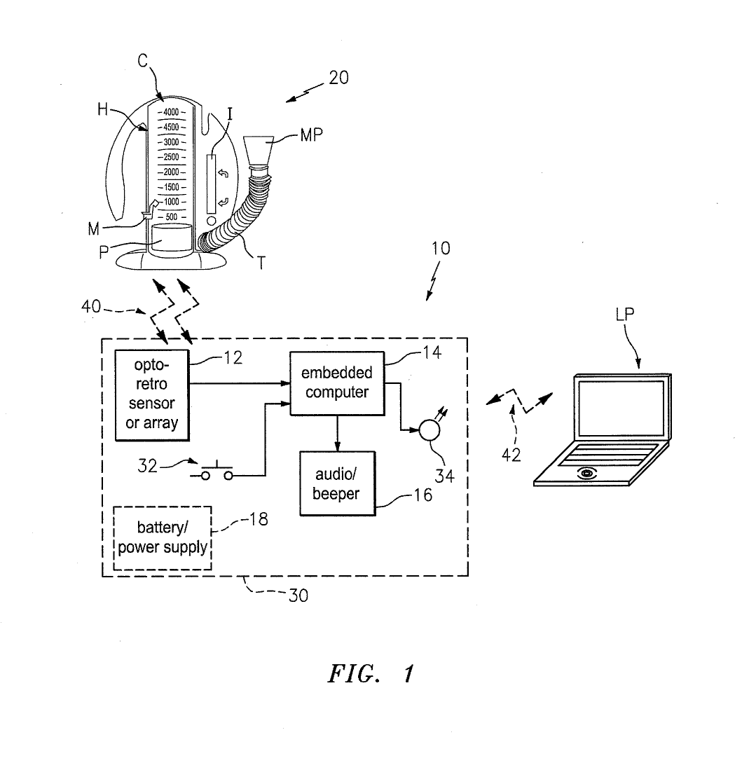

System 10, FIG. 1, includes at least one sensor 12, a processor 14 such as a microprocessor or embedded computer, and an alarm device 16. In this construction, the alarm device 16 includes a mechanism that produces an audible sound such as repeated beeps until session conditions are satisfied by the user. System 10 can be configured within a discrete package as indicated by dashed line 30 and discussed in more detail below, and includes a battery or other power supply 18 in this construction. System 10 further includes a mode control button 32 and a status or mode indicator light 34 such as one or more LEDs that are controlled by processor 14.

Sensor 12 senses movement of a piston P within a chamber C on an incentive spirometer 20 when a user inhales through mouth piece MP and flexible tubing T into chamber C defined by housing H of the incentive spirometer 20; the inhaled breath displaces a piston P within chamber C. In some constructions, the incentive spirometer 20 has an indicator I which provides inspiratory flow feedback to a patient or other user during inhalation. In one construction, signal 40 represents a modulated infrared signal utilized to detect movement of piston P as described below. A conventional marker M on housing H can be manually adjusted to visually indicate a desired minimum movement level for the piston P. Signal 42 represents wireless communication between system 10 and a computing device such as Bluetooth Low Energy transmission to and from a laptop LP. The laptop LP or other computing device typically executes data collection instructions as desired by a healthcare professional.

Sessions for a user to utilize the incentive spirometer 20 and the timing for sampling periods/wake-up rate for data acquisition from sensor 12 typically are set by a healthcare professional at a selected rate such as once an hour. In some constructions, adjustment of parameters in system 10 and/or laptop LP require a password, a special key or a special tool so that only a healthcare professional or an authorized person can make such adjustments.

At the start of a session, alarm device 16 begins beeping or generates another type of perceptible prompt to the user. In certain constructions, the alarm device 16 communicates with a smart phone, a tablet or other mobile device of a user to produce a vibration or other tactile sensation, a sound as an auditory prompt, and/or a visible message or visual cue to induce the user to begin the session. In another construction, the vibratory or tactile prompt is integrated into the device attached to the incentive spirometer.

In one construction, time-stamped data is stored in a storage medium such as a flash drive until required for upload. The processor 14 and/or laptop LP typically are maintained in “sleep mode” when possible to reduce power consumption.

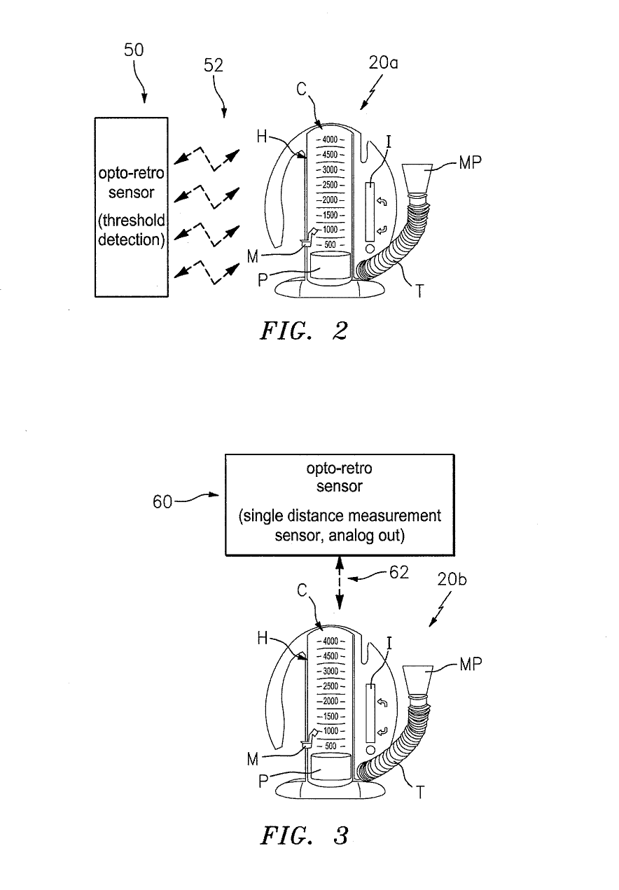

FIG. 2 is a schematic diagram of a sensor array 50 with multiple detectors utilized according to the present invention to detect piston movement in an incentive spirometer 20a. Signals 52 represent a plurality of readings from the multiple detectors. In one construction, the sensor array 50 is placed along the vertical axis of chamber C defined by housing H of the incentive spirometer 20a. As the piston P crosses each detector path, the microprocessor records approximate placement of the piston P with respect to the present and historic sensor data. This sensor configuration allows for tracking velocity of piston and lung function. This sensor configuration allows for identification of maximal inspiratory volume and achievement of target inspiratory volume. Longitudinal recording of sensor positioning, target inspiratory volumes achieved, enable for successive approximation of progression of lung volumes and function.

FIG. 3 is a schematic diagram of another sensor 60 utilized according to the present invention to detect vertical distance of the piston P from the sensor 60 mounted at the top of chamber C of the incentive spirometer 20b. In this construction, a single distance-measuring sensor is positioned at the top of the piston path. The sensor reports detected distance to the piston, which is utilized to calculate relative piston position. This construction may provide greater sampling resolution and performance history. This configuration allows for determination of precise inspiratory volume achievements. Tracked longitudinally, sequential inspiratory volume measures indicate progression of lung volumes and function.

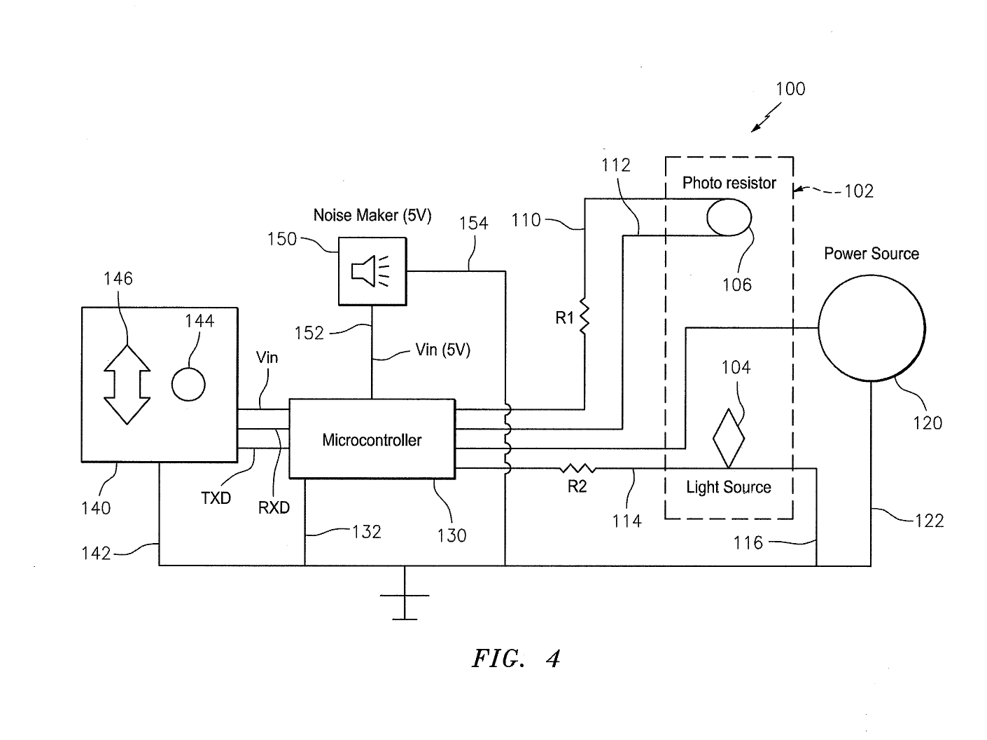

FIG. 4 is a schematic diagram of an alternative circuit design 100. A sensor 102 includes a source 104 of optical radiation, also referred to as light source 104, and a photo resistor 106 as a detector in this construction. Wires 110 and 112 connect photo resistor 106 to microcontroller 130, and may include one or more resistors R1. Light source 104 is connected directly to microcontroller 130 by wire 114, which may include one or more resistors R2, and is connected via wire 116 to power source 120 via wire 122 in one construction and, in other constructions, is connected to microcontroller 130 via wire 132 or through control unit 140 via wire 142. Control unit 140 includes an on/off switch 144 and a control 146 to enable an authorized user to increase or decrease the interval between sessions. Also included in this construction is a noise maker 150 that is controlled via wire 152 connected to the microcontroller 130 and is powered through wire 154 connected to power source 120.

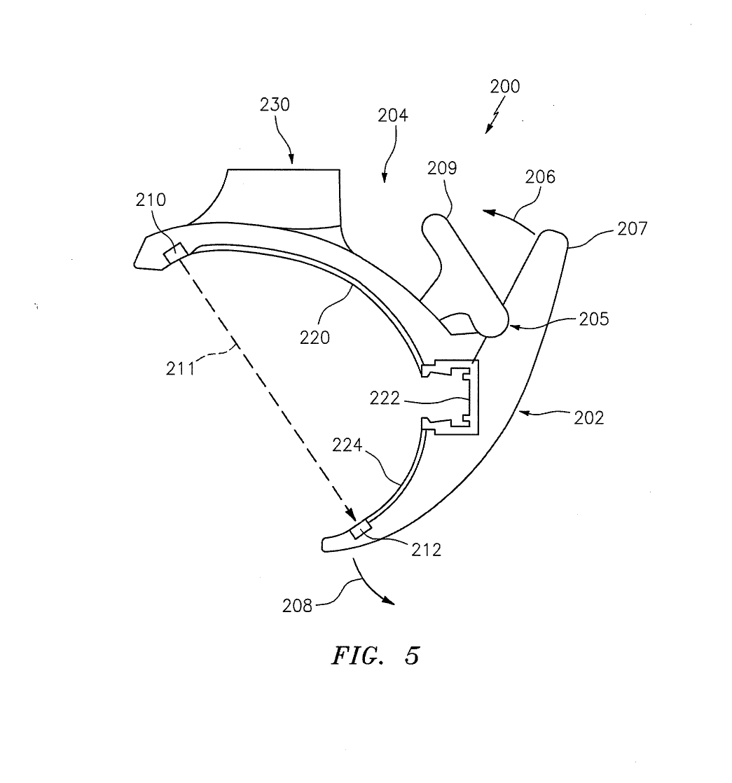

In one construction, circuit 100 represents the architecture of components within a clamp system 200 according to the present invention, FIG. 5. The clamp system 200 is removably placed on an incentive spirometer as desired over the cylindrical tube defining the chamber of the incentive spirometer. In one construction, gripping surfaces 220, 222 and 224 are similar in shape to marker M, FIG. 1. Clamp system 200, FIG. 5, includes a first portion 202 and a second portion 204 that are pivotally connected with each other at pivot hinge 205, which is preferably biased to a closed condition as illustrated. In this construction, projections 207 and 209 can be squeezed together to move portion 202 relative to portion 204 to an open condition as indicated by motion arrows 206 and 208, and thereby move one or more of gripping surfaces 220, 222 and 224 away from the housing of the incentive spirometer to assist initial placement or removal.

A light source 210 directs optical radiation 211 through the tube and a photo sensor 212 identifies disruption of the optical radiation 211 when the piston passes between the light source 210 and the detector 212. In some constructions, one or more of the gripping surfaces 220, 222 and 224 are formed from an optically opaque material, such as a high-friction rubber compound, which also serves to block extraneous light from interfering with readings by detector 212. One or more of a power source, a microcontroller, a control unit and an alarm device, such as components 120, 130, 140 and 150 of FIG. 4, contained within a housing 230, FIG. 5.

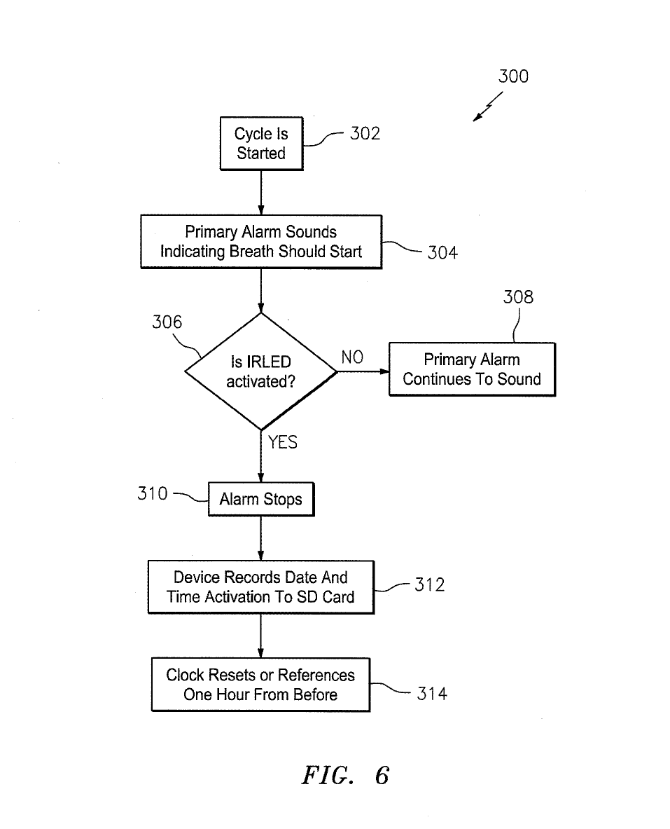

In some constructions according to the present invention, a microcontroller takes the voltage inputs of a sensor for incentive spirometer piston movement and then outputs a signal and records all events, as well as silences an alarm once compliance has been achieved. Flow chart 300, FIG. 6, represents the operation of one system according to the present invention, also referred to as an electromechanical medical device, or simply as a device, and methods of using same. A compliance cycle is started, step 302, and a primary alarm is activated, step 304, such as the sounding of a buzzer which indicates that a breath into the incentive spirometer should start. A light source such as an infrared LED is monitored, step 306. If the IRLED is not activated (i.e., if its beam is not interrupted) by sufficient movement of the piston, the primary alarm continues to sound as indicated by step 308. However, once the IRLED is activated, the alarm is stopped, step 310. The device records the date and time of IRLED activation on a SD card or other storage media, step 312, and a timer clock resets or begins counting the next hour or other selected time period, step. 314. The operation returns to cycle start, step 302, as long as power is supplied to the device or until it is altered by a healthcare professional.

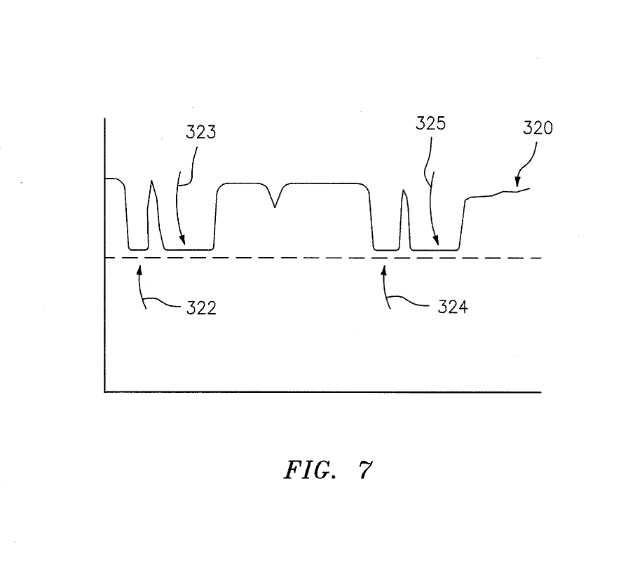

For incentive spirometers having chamber walls that are transparent to infrared (IR) light, the piston could be detected as it travels past the sensor. Given this signal state and history, the embedded computer can determine approximate placement of the piston along its travel axis. IR light emitters were considered due to wide availability and inexpensive cost of IR-specific sensors. To test chamber transparency to IR and to measure the effect of IR reflected back by piston movement, an IS chamber was placed within apparatus 600, FIGS. 10A-10E, having an IR LED and sensor, and observed oscilloscope output 320, FIG. 7, as the piston moved passed the IR LED and the sensor. Oscilloscope readings 320 drop as a piston passes up, as indicated by lower arrows 322 and 324, and drop down again as indicated by upper arrows 323 and 325. The change in voltages observed verified the use of IR as suitable optical radiation.

Hardware components of a typical system according to the present invention include selection of an incentive spirometer, a mechanical mount for detection equipment, an optical emitter, detection system interface electronics, embedded acquisition and recording microcontroller computer, network interface for logging data transfer, beeper for patient audio notification, mode configuration pushbutton, mode configuration LED indicator, power supply, data logging transfer and storage computer. Two modules of system software are utilized: software for an embedded acquisition computer and software for data download computer such as a laptop computer.

The embedded acquisition and recording microcontroller computer was considered—configuring it with a beeper (reminder alarm), an LED, and a button. This enables configuration of certain aspects of the device with the button, receive information via LED blink patterns, and use the beeper to indicate state to the clinician, patient, or other. For system 10, FIG. 1, data is gathered to local storage (embedded solid-state “flash” media), then the data can be uploaded manually via USB linkage to a host computer running some custom software. The embedded computer preferably is a low-power device, linkable to a software stack (a group of programmable software programs), cost efficient, and able to connect via USB—the Arduino 101 was selected for meeting these criteria.

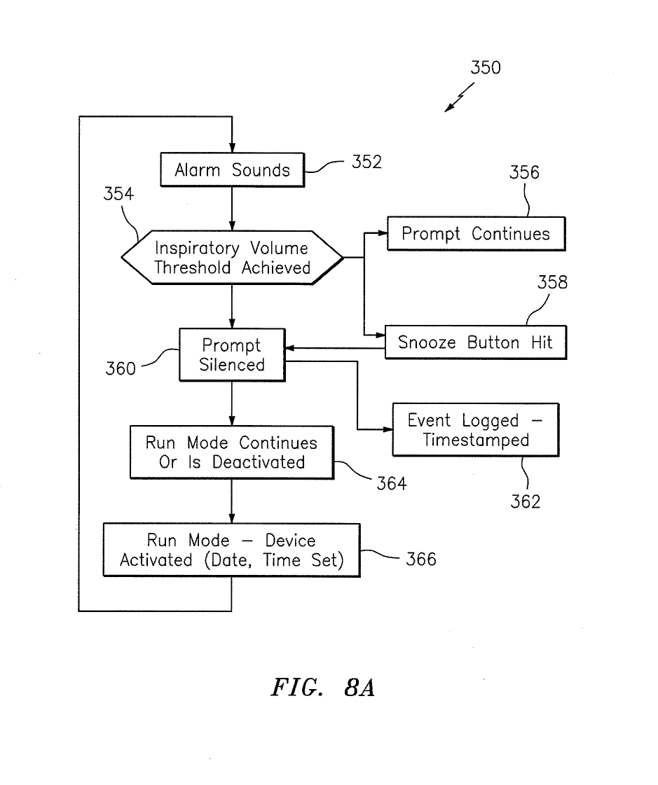

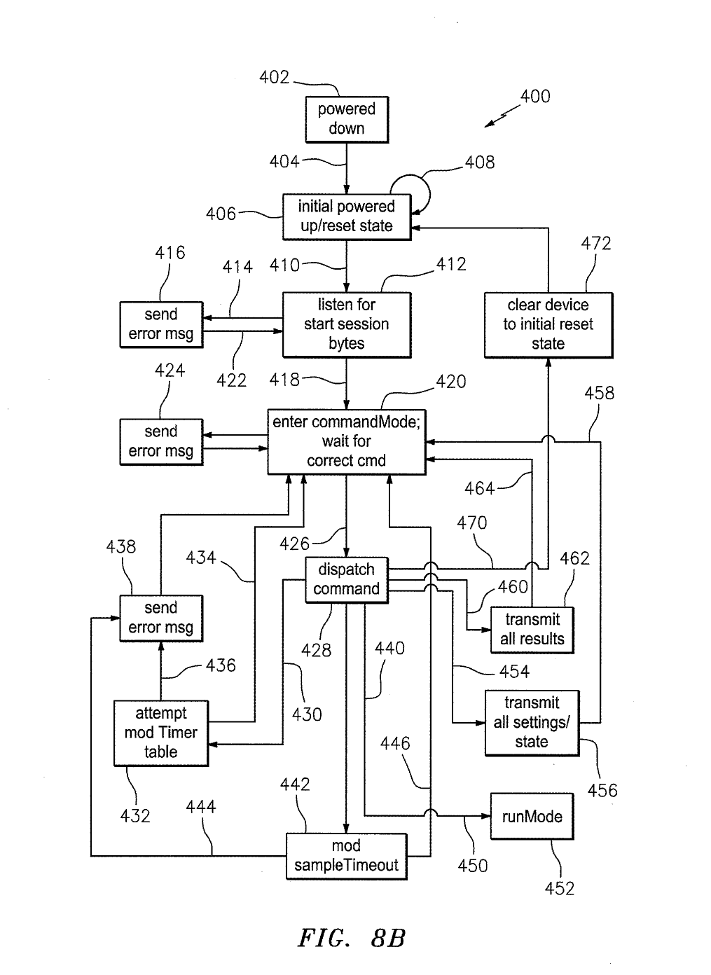

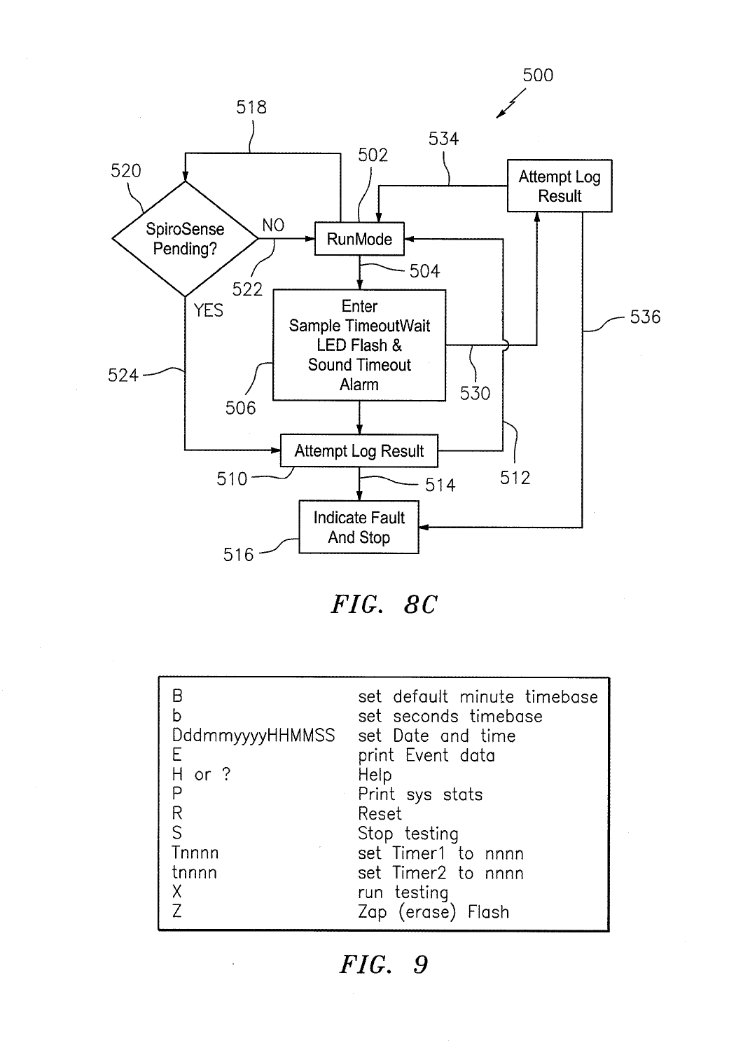

Operation of one construction according to the present invention is shown in flow chart 350, FIG. 8A. After an alarm sounds, step 352, it is determined whether inspiratory volume threshold is achieved, step 354. If not, the alarm prompt continues, step 356, unless the snooze button is depressed, step 358. If the volume threshold is achieved, or if the snooze button is depressed, then the prompt is silenced, step 360. Run mode continues or is deactivated, step 364, depending on system configuration. When run mode is reactivated and parameters such as date and time are set, step 366, the operation proceeds to step 352 when the next utilization of the incentive spirometer is scheduled.

FIGS. 8B and 8C schematically depict state control diagrams 400 and 500 which can be utilized in other constructions to program a host laptop and an embedded computer, respectively. The diagrams 400 and 500 are examples of guidance utilized to build appropriate software modules using a scripting system such as Python. Each state diagram 400, 500 indicates desired functional controls for certain systems and methods according to the present invention. An “arrow” refers to an action or a signal that is transmitted over a “wire”, that is, over a solid connection, in some constructions and, in other constructions, is transmitted wirelessly. For example, from a powered down condition, step 402 of FIG. 8A, the device is powered up, arrow 404, and an initial powered-up or reset state is achieved, step 406. Determination of USB connection is indicated by loop 408. After USB serial connection is achieved, arrow 410, the system listens for start session bytes, step 412. If incorrect bytes are received, arrow 414, an error message is sent, step 416, and the operation returns to step 412.

After correct bytes are received, arrow 418, the system enters “commandMode” and waits for the correct command, step 420. If incorrect bytes are received, arrow 422, then an error message is sent, step 424. When a good command is received, arrow 426, the command is dispatched, step 428. For example, “add Timer Command”, arrow 430, leads to an attempt to modify Timer table, step 432. If successful, arrow 434, the operation returns to step 420. If the attempt fails, arrow 436, then an error message is sent, step 438, and the operation then returns to step 420.

Similarly, a “modSampleTimoutCmd”, arrow 440, attempts to modify the sample Timeout, step 442. Failure, arrow 444, leads to generation of an error message, step 438, and the operation returns to step 420. A “runCmd”, arrow 450, leads to run mode, step 452. A “1stCmd”, arrow 454, leads to transmission of all settings and state, step 456; once complete, arrow 458, the operation returns to step 420. An “uploadCmd”, arrow 460, causes transmission of all results, step 462 and, once complete, arrow 464, operation returns to step 420. By comparison, a “resetCmd”, arrow 470, clears the device to its initial reset state, step 472, and the operation returns to step 406.

In diagram 500, FIG. 8B, run mode is initiated, step 502, and “nextTimeout”, arrow 504, leads to entering sample Timeout Wait, LED flash & sound timeout alarm, step 506. For timeout, arrow 508, a log result is attempted, step 510; if successful, arrow 512, then the operation returns to step 502. If the log attempt fails, arrow 514, then the system indicates “fault” and stops, step 516.

If incentive spirometer threshold is sensed, arrow 518, then it is determined if “spiroSense” is pending, step 520. If no, arrow 522, the operation returns to runMode, step 502; if yes, arrow 524, the operation proceeds to attempt log, step 510. From step 506, if incentive spirometer threshold is sensed, arrow 530, log result is attempted, step 532; if successful, arrow 534, the operation returns to runMode, step 502; if it fails, arrow 536, the operation proceeds to step 516 and stops.

FIG. 9 is an example of a listing of command lines 550 for firmware in one system according to the present invention. Device commands were created to allow for customization of beeper sound frequency and duration. All events—inspiration above threshold, snooze, alarm sounding—are time-stamped and recorded. Device commands are set up and data collection is started and stopped using the host computer input.

To ensure patients were able to hear the alarm, a buzzer 642, FIGS. 10E and 11 (RadioShack 3 VDC Mini Buzzer in one construction), was selected with a tone (300-500 Hz) and decibels (75 dB) that would be perceptible to elderly patients with hearing loss. The microcontroller along with the IR light emitter and sensor was assembled.

The apparatus represented by circuit design 100, FIG. 4, was effective at capturing inspiratory events in ambient room light. However, in a repeat test adjacent to a window in direct, high-intensity sunlight the light signal noise interference prevented inspiratory event capture. To test light-noise reduction strategies, the incentive spirometer was covered in black electrical tape, exposed the apparatus to direct, high-intensity sunlight and were able to reliable capture inspiratory events. Housing (using CAD) was designed to cover the incentive spirometer chamber and electronic components, as described in more detail below in relation to FIGS. 10A-10E. The housing 602 benefitted from a base to receive and secure new incentive spirometers in and an adjustable volume sensor positioner. The base 604 also added some weight to the devices to help keep the apparatus upright to avoid false positive inspiration readings from an incentive spirometer being turned onto its side accidentally or intentionally. The component parts were 3D printed using fused deposition modelling in black (light blocking) acrylonitrile butadiene styrene—a polymer with a balance of shininess, rubbery feel, and toughness to be appropriate for repeated use in a clinical study.

FIGS. 10A-10E are schematic illustrations of an apparatus 600 with an opaque housing 602 for one system according to the present invention. In one construction, the assembled instrument 600 works with an interchangeable incentive spirometer IS, shown in phantom, an Arduino 101 controller computer 610, FIG. 10E, within an electronics housing 612, and an optical sensor and IRLED unit 620, FIG. 10C, within adjustable positioner 630, movably mounted to vertical post 632, to detect piston travel within the IS chamber. Unit 620 is mounted within positioner 630 on a vertical support 622 in this construction having support struts 624 and 626.

Opaque housing 602 defines a plurality of view ports which are open in this construction and, in other constructions, can have optically transparent or translucent plates to enable viewing of a device such as an incentive spirometer IS. Graduation marks 605 on the incentive spirometer IS are visible through port 606, FIG. 10A, to enable viewing of piston travel. Indicators 607 such as breath flow rate are visible through port 608. A port 609, FIG. 10D, provides optical access for the light source and detector unit 620 within positioner 630, FIG. 10C. Additional viewing ports such as optional port 611, shown in phantom in FIG. 10D, are provided in other certain constructions to enable viewing or other access within opaque housing 602 as desired.

A conduit 628, FIG. 10C, positioned between struts 624 and 626 enables wired connection to the controller 610, FIG. 10E, for signal and power. A single button 640 serves as a ‘snooze’ button which is also connected to controller 610. A power cable 660 is connectable to an external controller power supply 662 when apparatus 600 does not carry a self-contained power source, or to periodically replenish an internal power source such as a rechargeable battery. A separate host laptop 670, FIG. 11, serves as a workstation 670, and a USB cable 668 connects the Arduino 101 computer 610 to the workstation 670, as shown schematically in FIG. 11. The Arduino microcontroller is configured using the workstation.

The workstation 670, FIG. 11, is used to configure test settings; all settings can be modified to the desired experimental design. Test settings include timer alarm frequency (e.g., every hour) and duration of reminder prompt (e.g., bell sound 1 minute). The Arduino 610 records and time stamps (date and military time including seconds) every event including start testing mode and inspiratory events. Snooze and sleep functions are optional. Time-stamping and tracking data usage is useful for clinical monitoring of both incentive spirometer frequency of use and inspiratory volume achievements.

When the device is put into testing mode, and the reminder alarm sounds or generates other perceptible indicia of alarm, the device indicates via the perceptible prompt that the patient should use the incentive spirometer. Immediately as the reminder alarms, a patient can: (1) do nothing and allow to the prompt to timeout; (2) push a snooze button, which will silence the alarm, or (3) use the incentive spirometer, which will also silence the indicator alarm if the patient hits or surpasses the IR light threshold. The system keeps track of what time each timer starts, and what time a snooze or inspiratory event happens. The system records all inspiratory events, both following and independent of timers. The system logs this data.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature.

It is the intention, therefore, to be limited only as indicated by the scope of the claims appended hereto. Other embodiments will occur to those skilled in the art and are within the following claims.

What is claimed is:

1. A method for configuring an incentive spirometer to interact with a user during a series of sessions, comprising:
selecting an incentive spirometer having a housing defining a chamber and a piston that is movable within the chamber by user inhalation;
mounting a sensor to detect movement of the piston;
generating a perceptible prompt to a user to encourage use of the incentive spirometer at preselected intervals relative to the series of sessions; and
deactivating the perceptible prompt only after the sensor detects that the piston has at least reached a preselected threshold.

2. The method of claim 1 wherein the perceptible prompt includes at least one of an audible sound, a visual prompt, and a vibratory tactile prompt.

3. The method of claim 1 wherein generating the perceptible prompt includes activating a prompt on a remote device.

4. The method of claim 3 wherein the remote device is a mobile device capable of being carried by the user.

5. The method of claim 1 further including storing detected piston movement in a storage medium as retrievable data.

6. The method of claim 1 wherein the sensor is removably mounted to the incentive spirometer.

7. The method of claim 6 wherein the sensor is integrated into a removable clamp.

8. The method of claim 1 wherein the perceptible prompt is deactivated only after the user breathes with sufficient inspiratory target volume using the incentive spirometer.

9. The method of claim 1 further including placing an opaque housing over at least a portion of the incentive spirometer housing.

10. A system to enhance compliance for a user of an incentive spirometer having a housing defining a chamber and a piston that is movable within the chamber by user inhalation, comprising:
a sensor mountable on the incentive spirometer to detect movement of the piston;
an alarm device for generating a perceptible prompt to the user to encourage use of the incentive spirometer; and
a processor capable of periodically activating the alarm device and then deactivating the alarm device only after the sensor detects that the piston has at least reached a preselected threshold.

11. The system of claim 10 wherein the alarm device includes a mechanism to generate at least one of an audible sound, a visual prompt, and a vibratory tactile prompt as the perceptible prompt.

12. The system of claim 10 further including the incentive spirometer.

13. The system of claim 10 wherein at least one of the processor and the alarm device activates a perceptible prompt on a remote device.

14. The system of claim 13 wherein the remote device is a mobile device capable of being carried by the user.

15. The system of claim 10 further including at least one storage medium to store detected piston movement as retrievable data.

16. The system of claim 10 wherein the sensor is removably mountable to the incentive spirometer.

17. The system of claim 16 wherein the sensor is integrated into a removable clamp.

18. The system of claim 10 wherein the perceptible prompt is deactivated only after the user breathes with sufficient inspiratory target volume using the incentive spirometer.

19. The system of claim 10 further including an opaque housing that covers at least a portion of the incentive spirometer housing.

20. The system of claim 19 wherein the sensor is capable of detecting optical radiation transmitted through the housing of the incentive spirometer, and the opaque housing is secured to a support base and is positioned to shield at least a portion of environmental optical radiation surrounding the incentive spirometer housing.
